(19) United States
(12) Patent Application Publication
(10) Pub. No.: US 2015/0342787 A1
(43) Pub. Date: Dec. 3, 2015

(54) INVASIVE MEDICAL DEVICE DRESSING AND METHOD OF APPLICATION

(71) Applicant: Valarie Williams HAISLEY, Central, SC (US)

(72) Inventor: Valarie Williams HAISLEY, Central, SC (US)

(21) Appl. No.: 14/725,417

(22) Filed: May 29, 2015

Related U.S. Application Data
(60) Provisional application No. 62/005,088, filed on May 30, 2014.

(51) Int. Cl. A61F 13/02

(52) U.S. Cl. CPC A61F 13/0206; A61F 2013/00182

(57) ABSTRACT

An invasive medical device dressing is provided. The dressing includes an absorbent foam layer and an adhesive film. The absorbent foam layer is applied over a portion of the patient's skin where an insertion site is formed for an invasive medical device. The adhesive film is secured over the foam layer so that the foam layer is sandwiched in between the skin and the adhesive film. The adhesive film secures the absorbent foam layer to the patient's skin.

### CROSS-REFERENCE TO RELATED APPLICATION

This application claims the benefit of priority of U.S. provisional application No. 62/005,088, filed May 30, 2014, the contents of which are herein incorporated by reference.

### BACKGROUND OF THE INVENTION

The present invention relates to a medical dressing and, more particularly, to an invasive medical device dressing and method of application.

Transparent film adhesive dressings are traditionally used over invasive medical devices such as vascular access devices. Examples of vascular access devices include central vascular access devices and peripheral vascular access devices i.e., non-tunneled percutaneous catheters, tunneled catheters, implanted ports, hemodialysis catheters, peripherally inserted central line catheters (PICCs), arterial lines, peripheral intravenous catheters, and extended length catheters.

Transparent film adhesive dressings are unable to adhere over invasive medical devices when excess moisture or drainage occurs, which is typically caused by diaphoresis, skin erosion, or bleeding present on the skin's surface immediately surrounding the insertion site. The adhesive properties of transparent film dressings do not promote adhesion in the presence of moisture because the adhesive dressings do not have the capability to absorb the moisture away from the skin and into the dressing. When being used as a dressing for invasive medical devices, the inability of transparent film adhesive dressings to adhere to the skin in the presence of excessive moisture or drainage creates a condition in which patients are at higher risk for acquiring an infection.

The accumulation of moisture and drainage under transparent film adhesive dressings renders the dressings ineffective in providing a physical barrier to pathogens. Therefore, unscheduled and frequent dressing changes must occur. Unscheduled and frequent dressing changes that occur before the end wear time of the dressing leads to skin erosion surrounding the invasive medical device insertion site. When any type of dressing is removed from the skin, a superficial layer of skin cells is removed from the skin. As long as the dressing is able to be worn for the intended wear time, skin cell regeneration and healing will occur on the skin's surface. However, if frequent and unscheduled dressing changes occur before the wear time of the dressing has been completed, the skin cells have not had time to regenerate and heal. This leads to skin erosion. Eroded skin becomes moist skin. Moist skin renders transparent film adhesive dressings incapable of adhering to the skin, and thus beginning the cycle of frequent, unscheduled dressing changes. The presence of skin erosion also leads to a higher risk of infection because the skin barrier is broken and thus incapable of providing protection from the invasion of pathogens.

As can be seen, there is a need for an improved dressing for invasive medical devices.

### SUMMARY OF THE INVENTION

In one aspect of the present invention, a method of applying a dressing to a patient with an invasive medical device comprises: applying absorbent foam layer over a portion of the patient's skin comprising an insertion site of the invasive medical device; and securing an adhesive film to the patient's skin over the absorbent foam layer, thereby securing the absorbent foam layer to the patient's skin.

In another aspect of the present invention, an invasive medical device dressing comprises: an absorbent foam layer comprising a window located in either the center or offset formed therethrough; and an adhesive film formed to secure the absorbent foam layer to a patient's skin.

These and other features, aspects and advantages of the present invention will become better understood with reference to the following drawings, description and claims.

### BRIEF DESCRIPTION OF THE DRAWINGS

FIG. 1 is a perspective detail view of an embodiment of the present invention shown in exemplary usage;

FIG. 2 is a perspective view of an embodiment of the foam layer of the present invention;

FIG. 3 is an exploded view of an embodiment of the present invention;

FIG. 4 is a section detail view of the present invention along line 4-4 in FIG. 1;

FIG. 5 is a perspective view of an alternate embodiment of the foam layer of the present invention; and

FIG. 6 is a perspective view of an alternate embodiment of the present invention shown in use.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

The present invention includes an invasive medical device dressing including a hydrophilic absorbent foam dressing secured to a patient by a transparent film adhesive. The present invention serves as an invasive medical device securement dressing. The application of hydrophilic absorbent foam dressing that is capable of wicking moisture away from the skin's surface and drawing the moisture into the dressing is thereby capable of adhering in the presence of moisture. The absorptive and adherence properties of a foam dressing allow the dressing to be worn for the designated wear time. The use of a transparent adhesive film placed on top of the absorbent foam dressing provides a high degree of dressing occlusiveness needed for invasive medical devices. The use of a hydrophilic absorbent foam dressing covered and secured by a transparent film adhesive decreases the risk of infection due to the absorption of moisture from the skin and into the foam dressing thus preventing skin erosion and leaving the skin barrier intact. Also, because the absorbent foam dressing is able to adhere to the skin in the presence of moisture or drainage, the risk of infection is decreased because the insertion site and the skin immediately surrounding the insertion site remains adequately covered.

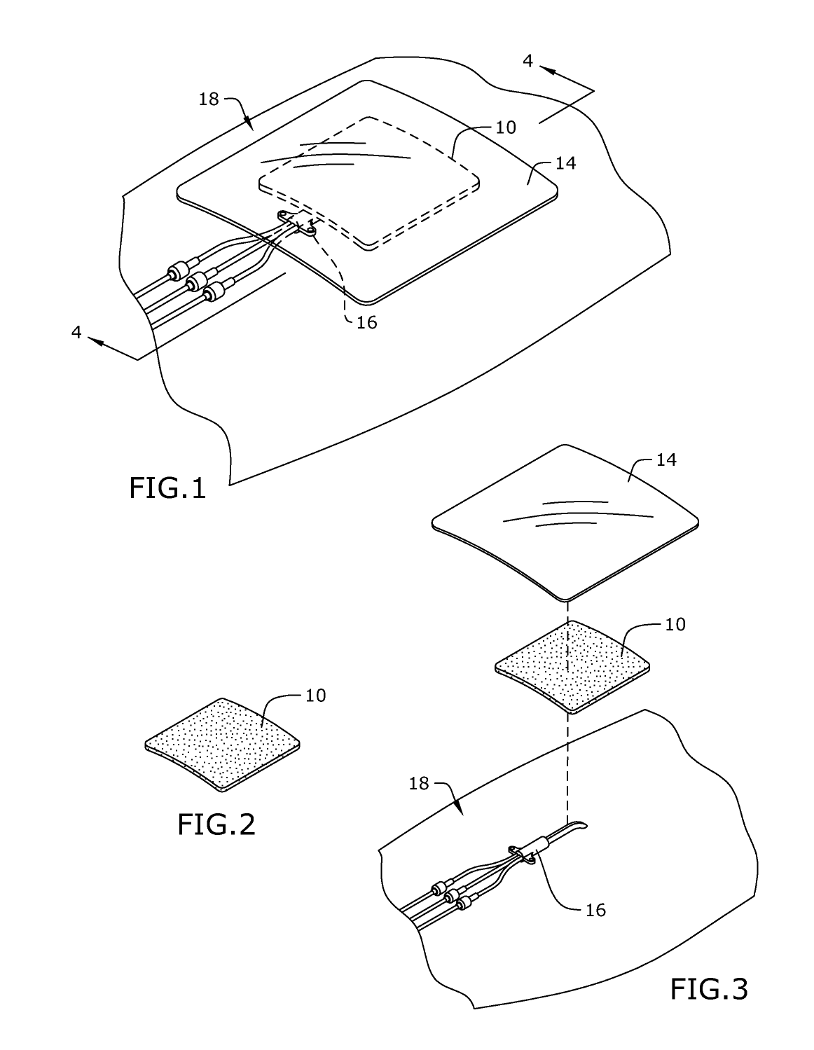

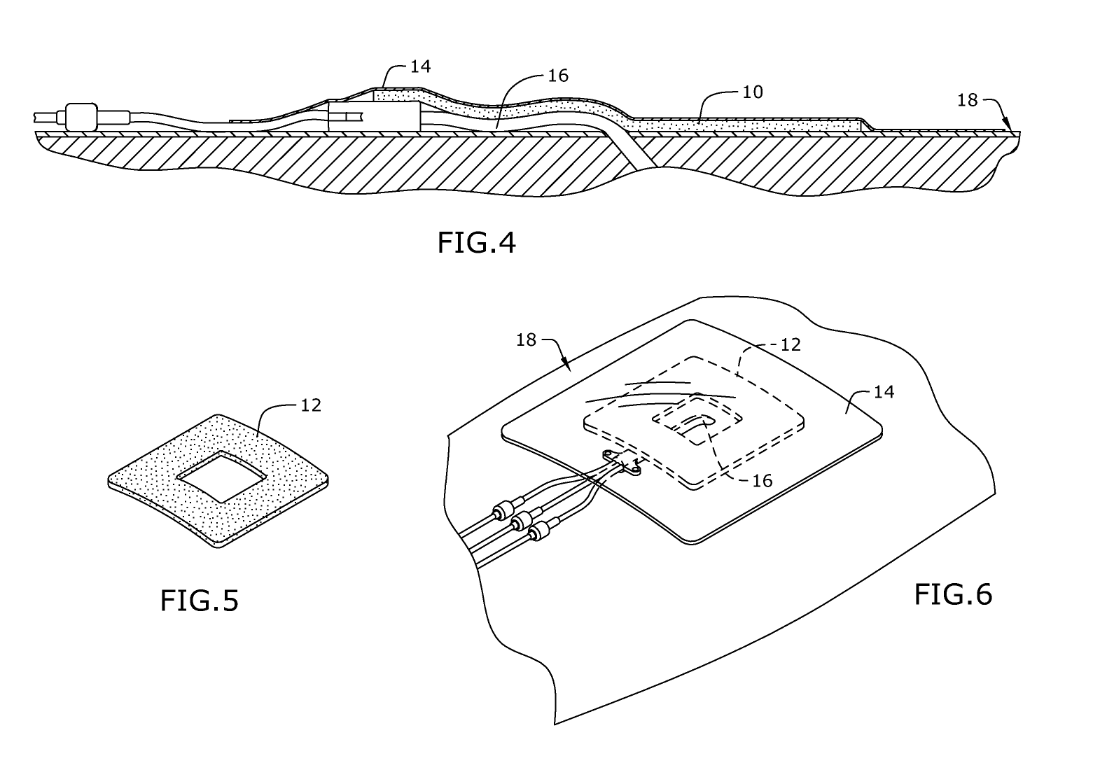

Referring to FIGS. 1 through 6, the present invention includes an invasive medical device dressing. The dressing includes an absorbent foam layer 10 and an adhesive film 14. The absorbent foam layer 10 is applied over a portion of the patient's skin 18 where an insertion site is formed for an invasive medical device 16. The adhesive film 14 is secured over the foam layer 10 so that the foam layer 10 is sandwiched in between the skin 18 and the adhesive film 14. The adhesive film 14 secures the absorbent foam layer to the patient's skin 18.

The adhesive film 14 may be a transparent tape. The adhesive film 14 may include a larger perimeter than the absorbent foam layer 10. Therefore, the edges of the adhesive film 14 may extend past the edges of the absorbent foam layer 10 and secure directly to the patient's skin 18.

In certain embodiments, the present invention may include a windowed absorbent foam layer 12. The window may be formed by an inner edge of the windowed absorbent foam layer 12 and may provide a viewing port to see the insertion site of the invasive medical device 16. This allows the clinician to monitor the vascular access insertion site. However, if bleeding is occurring from the insertion site or the insertion site does not need to be visible, then the absorbent foam dressing 10 without a window may be used.

The hydrophilic polyurethane foam and the moisture vapor permeable polyurethane transparent film adhesive layer may be pre-fabricated and manufactured in advance as one unit in a variety of foam dressing sizes, which may include a variety of window sizes to accommodate the intended use and positioning. The size of the dressing may be made to accommodate the neonatal, pediatric, and adult population. The window in the foam may be cut in different sizes to accommodate the different catheter sizes of vascular access devices.

The pre-fabricated dressing may be manufactured through a commercial process in which polyurethane foam and a moisture vapor permeable polyurethane transparent film adhesive layer are be combined as one product. During the manufacturing process, a window may be cut either in the center of the foam or offset if needed, depending on the requirement of the invasive medical device. The transparent film adhesive layer is integrated and adherent to the top outer surface of the foam. The dressing may be sterilized per industry's standards and then labeled and packaged in individual packages.

During application, the foam and adhesive film combination may be removed from a packaging. The clinician applying the dressing may use sterile techniques to apply the dressing over the invasive medical device. On the foam side of the adhesive film that is to be applied to the patient, there may be a liner. The clinician may remove this liner and then apply the foam over the invasive medicial device and onto the patient's skin surrounding the invasive medical device. The transparent film adhesive layer covers the entire foam dressing and overlaps onto the patient's skin. If a window is present, the window is placed over the insertion site. The clinician applies gentle pressure on the transparent film adhesive layer to ensure adherence.

Another method of producing this dressing is for the clinician to make the dressing at the bedside. Sterile techniques and sterile supplies may be used. Scissors may be used to cut a window in the center or offset through the foam dressing. This allows visualization and monitoring of the invasive medical device insertion site. If visualization of the insertion site is not needed, then a non-windowed foam dressing may be used. The clinician cuts the foam to the size desired and cuts the window to the desired size based on the size of the invasive medical device. The foam dressing is be placed over the insertion site and onto the patient's skin. A moisture vapor permeable polyurethane transparent film adhesive dressing is then be applied over the foam and allowed to overlap onto the patient's skin. The purpose of the transparent film adhesive dressing is to secure the foam dressing and to provide the high degree of occlusiveness needed when covering invasive medical devices. The clinician may apply gentle pressure to the transparent film to ensure adherence.

The hydrophilic absorbent foam dressing of the present invention is able to absorb excess moisture or drainage on the skin's surface typically caused by diaphoresis, skin erosion, or bleeding. The ability of the dressing to absorb moisture from the skin allows the dressing to adhere to the skin even in the presence of moisture for the dressing designated wear time. A transparent film dressing covers the windowed or non-windowed foam dressing and overlaps onto the skin surrounding the foam dressing. The transparent film dressing securely covers the foam dressing over the vascular access device. The transparent film adhesive provides the high degree of occlusiveness needed for vascular access devices and overlaps onto the skin to provide a healthy margin.

The use of this invention allows dressing adherence for the designated wear time. A hydrophilic absorbent foam dressing will decrease the need for unscheduled, frequent dressing changes and thus contribute to the maintenance of skin integrity. The decrease in the number of dressing changes needed decreases the incidence of skin injury and erosion. The properties of the foam dressing also aid in the healing of any injured or eroded skin present on the skin.

In addition, the present invention may function as a primary or secondary dressing for superficial, partial thickness and full thickness wounds, such as vascular ulcers, neuropathic ulcers, pressure ulcers, and surgical approximated or non-approximated wounds.

It should be understood, of course, that the foregoing relates to exemplary embodiments of the invention and that modifications may be made without departing from the spirit and scope of the invention as set forth in the following claims.

What is claimed is:

1. A method of applying a dressing to a patient with an invasive medical device comprising:
applying an absorbent foam layer over a portion of the patient's skin comprising an insertion site of the invasive medical device; and
securing an adhesive film to the patient's skin over the absorbent foam layer, thereby securing the absorbent foam layer to the patient's skin.

2. The method of claim 1, wherein the adhesive film is a transparent moisture vapor permeable polyurethane dressing.

3. The method of claim 2, wherein the absorbent foam layer comprises a window formed therethrough.

4. The method of claim 3, wherein the absorbent foam layer is positioned so that the insertion site of the invasive medical device is visible through the window.

5. The method of claim 1, wherein the adhesive film covers an entire perimeter of the absorbent foam layer.

6. The method of claim 1, wherein the invasive medical device is a vascular access device.

7. An invasive medical device dressing comprising:
an absorbent foam layer comprising a window formed therethrough; and
an adhesive film formed to secure the absorbent foam layer to a patient's skin.

8. The invasive medical device dressing of claim 7, wherein the adhesive film comprises a larger perimeter than the absorbent foam layer.

9. The invasive medical device dressing of claim 7, wherein the adhesive film is a transparent moisture vapor permeable polyurethane dressing.

10. The invasive medical device dressing of claim 7, wherein the absorbent foam layer is comprised of a hydrophilic polyurethane foam.
